(19) United States
(12) Patent Application Publication
(10) Pub. No.: US 2024/0308900 A1
(43) Pub. Date: Sep. 19, 2024

(54) Dental Material With Pigment

(71) Applicant: Ivoclar Vivadent AG, Schaan (LI)

(72) Inventors: Corina Serban, Lindau (DE); Urs Bolle, Koblach (AT); Christin Wildner, Vilters (CH); Frank Rothbrust, Röns (AT); Sebastian Krolikowski, Lachen (CH); Christian Ritzberger, Grabs (CH)

(21) Appl. No.: 18/605,732

(22) Filed: Mar. 14, 2024

(30) Foreign Application Priority Data
Mar. 16, 2023 (EP) 23162377.8

(51) Int. Cl. C03C 1/04; A61C 13/08; A61C 13/083; C01G 25/00; C03C 8/20; C03C 10/16

(52) U.S. Cl. CPC C03C 1/04; C01G 25/006; C03C 8/20; C03C 10/16; A61C 13/082; A61C 13/083; C01P 2002/34; C01P 2002/52; C01P 2004/61; C01P 2004/62; C01P 2004/64; C01P 2006/63

(57) ABSTRACT

A dental material comprising a pigment, wherein the pigment contains Al, Cr and one or more of Y, La and lanthanides. The dental material allows mimicking the red coloration of natural teeth and natural oral mucosa. Also provided is the use of the dental material and the pigment and starting compositions for making the pigment in the preparation of a dental restoration. Furthermore, a process for preparing a dental restoration and a process for preparing the pigment is provided.

### CROSS-REFERENCE TO RELATED APPLICATIONS

This application claims priority to EP 23162377.8, filed on Mar. 16, 2023, the disclosure of which is incorporated herein by reference in its entirety.

### TECHNICAL FIELD

The invention relates to a dental material containing a pigment.

### BACKGROUND

Dental restorations, which can e.g., be made of glasses, glass ceramics or oxide ceramics, are subject to high demands in terms of their mechanical properties. It is also desirable to give dental restorations an appearance that is as close to nature as possible. The aim is to imitate the translucency properties of natural tooth material and also to achieve the best possible color match of the dental restoration with the remaining natural teeth and, if necessary, with the color of the oral mucosa and in particular the gingiva. When imitating the color of natural teeth and natural gingiva, it is in particular necessary to achieve a yellow and a red coloration in the materials.

There are different approaches to coloring dental materials red.

In glasses and glass ceramics, a red coloration is achieved in particular by particles of colloidal gold, wherein tin often is also added to the glass as a reducing agent. Glass that is colored red in this manner is also known as gold ruby glass. Particles of colloidal gold can, for example, be added to glazes or layering materials to achieve a red color of glasses or materials used in the preparation of dental layering restorations. However, only a limited number of different red shades can be realized with the particles of colloidal gold. Another disadvantage is that the particles of colloidal gold have very limited heat stability at the temperatures used for the preparation of dental restorations. Therefore, at high temperatures, such as 1000° C. and higher, there is a weakening or even a complete loss of the red coloring effect. For this reason, for example, high-melting glasses intended for processing at temperatures above 1000° C. cannot be colored red with particles of colloidal gold.

Glasses and glass ceramics can also be colored red using cadmium pigments, such as cadmium sulfoselenide Cd(S, Se) in particular. However, due to their high toxic potential, cadmium pigments must not be used in the manufacture of dental restorations.

### SUMMARY

The problem of the invention is therefore to provide dental materials with different red colorations. The red colors should, for example, be able to imitate the red colors of the gingiva or of natural teeth. In addition, the red colors should have a high temperature stability so that the dental materials can be exposed, for example, to the high temperatures typically used in the manufacture of dental restorations. The dental material should also allow easy and fast processing and in addition be inexpensive. A health hazard to the persons entrusted with the preparation and to the patients should also be avoided.

This object is solved by the dental material described herein and the use described herein. The invention is also directed to the process for preparing a dental restoration described herein and the process for preparing the pigment described herein.

### DETAILED DESCRIPTION

The dental material according to the invention is characterized in that it contains a pigment, wherein the pigment comprises Al, Cr, and Z, and Z is selected from the group consisting of Y, La, lanthanides and mixtures thereof.

With the dental material according to the invention, dental restorations can be provided with colors that are perceived as colors of natural teeth or of the oral mucosa. In particular, the dental material according to the invention allows imitating the red coloration of natural teeth and even of the natural oral mucosa when preparing dental restorations.

It has been surprisingly found that the pigments in the dental material are stable even at the high temperatures typically used for preparing dental restorations from glasses, glass ceramics or feldspar ceramics. Therefore, it is particularly advantageous to use the dental material according to the invention for the preparation of dental restorations.

It has been found that the high temperature stability is also present in the respective microstructure of the dental material, such as the microstructure of the glass ceramic or the feldspar ceramic. It has also been found that the dental material according to the invention has mechanical properties equivalent to the corresponding conventional dental materials that do not contain the pigment. This is surprising because the fine distribution of pigments in dental materials often results in undesirable effects on the microstructure. In particular, it can lead to the formation of local eutectics and a deterioration of the mechanical properties of the dental restoration or undesirable effects during heat treatments.

It has also been shown that the dental materials according to the invention allow an intense red color to be achieved in a cost-effective process. In the case that the dental material is colored in a single red color by using the pigment, any possible subsequent steps to create a natural multicoloration are facilitated.

The dental material according to the invention is also particularly advantageous in terms of toxicity risk and biocompatibility, as it poses no health risk to the persons entrusted with the processing of the dental material and to the patients.

According to the invention, the terms “color” and “colored” relate to the color value of a material. Color values can be characterized by the L*a*b* value using a spectrophotometer according to DIN 6174 or by a shade guide commonly used in the dental industry. The terms “red colors” and “red shades” relate to colors with a positive a*value in the L*a*b* color space.

It is preferred that the pigment comprises Al and Z in a molar ratio of 0.7:1 to 1:0.7, preferably 0.8:1 to 1:0.8 and particularly preferably 0.9:1 to 1:0.9.

In a preferred embodiment, the pigment comprises Z, Al and Cr in a molar ratio corresponding to the formula ZₓAl_{2-x-y}Cr_{y}O₃, wherein x is 0.8 to 1.2, preferably 0.9 to 1.1, in particular 0.95 to 1.05 and particularly preferably 1.00, and y is 0.001 to 0.5, preferably 0.005 to 0.25 and in particular 0.01 to 0.1.

In a further preferred embodiment, the pigment additionally contains Ca.

In a particularly preferred embodiment, the pigment has a composition corresponding to the formula ZₓAl_{2-x-y}Cr_{y}O₃, where x is from 0.8 to 1.2, preferably from 0.9 to 1.1 and in particular from 0.95 to 1.05 and particularly preferably 1.00, and y is from 0.001 to 0.5, preferably from 0.005 to 0.25 and in particular from 0.01 to 0.1.

In another preferred embodiment, the pigment has a composition corresponding to the formula ZₓAl_{2-x-y}Cr_{y}O₃, wherein Z is selected from the group consisting of yttrium, La and lanthanides, x is 1 and y is 0.001 to 0.5. In this case, the pigment has the formula ZAl_{1-y}Cr_{y}O₃, wherein Z is selected from the group consisting of yttrium, La and lanthanides and y is 0.001 to 0.5, preferably 0.005 to 0.25 and particularly preferably 0.01 to 0.1. It is further preferred that the pigment has a composition substantially corresponding to the formula YAl_{0.97}Cr_{0.03}O₃.

The molar ratio of Z, Al and Cr in the pigment as well as the composition of the pigment can be determined, for example, by inductively coupled plasma optical emission spectroscopy (ICP-OES), inductively coupled plasma mass spectrometry (ICP-MS) or atomic absorption spectrometry (AAS).

In a preferred embodiment, the main crystal phase of the pigment has a perovskite crystal structure. In this context, the term “main crystal phase” refers to the crystal phase that has the highest mass fraction of all the crystal phases present in the pigment.

The perovskite crystal structure can also be distorted. The term perovskite crystal structure also covers doped perovskite crystals. The pigment can additionally have other crystal phases, such as a garnet structure or a monoclinic crystal structure. For example, when Z is yttrium, the pigment can have yttrium-aluminum perovskite (YAP) as the main crystal phase and yttrium-aluminum garnet (YAG) and/or a monoclinic yttrium-aluminum crystal phase (YAM) as additional crystal phases.

The crystal phases present in the pigment can be determined by X-ray diffraction (XRD) analysis. Quantification of the crystal phases can be carried out, in particular, by the Rietveld method.

It is further preferred that Z is selected from the group consisting of Y, Er, Pr, Gd, Dy, Eu, Nd, Yb, Ho and Tm. It has been shown that a particularly intense red color with high a*values can be achieved with these pigments.

In a preferred embodiment of the dental material, the pigment has an average particle size d₅₀, determined according to ISO 13320, of 0.05 to 50 μm, in particular 0.1 to 25 μm and particularly preferably 0.5 to 15 μm. Particle sizes in these ranges have been found to be particularly advantageous for the mechanical properties of the dental material. In particular, it has been found that the use of small particles can be advantageous for preparing dental restorations with a particularly high strength.

In a preferred embodiment, the pigment has a color that has an a*value of at least 7, in particular at least 10. It is particularly preferred that, in addition, the b* value is 5 to 30, in particular 10 to 30, and the L* value is 40 to 65, in particular 45 to 60.

In a further preferred embodiment, the pigment has a color that has an a*value of 7 to 40, preferably 10 to 32, a b* value of 5 to 30, and an L* value of 40 to 85. It is particularly preferred that the pigment has a color that has an a*value of 25 to 32, a b* value of 15 to 22 and an L* value of 40 to 60.

It has surprisingly been found that pigments which are perceived to have a brown color when used as powder or in concentrated solution can also produce colors in dental materials that can be used to mimic the red color of natural teeth or the oral mucosa.

Preferably, the dental material is present in the form of a powder, a blank, or a dental restoration.

Thus, the term “dental material” relates to any material, no matter what processing step, that eventually serves at least as part of a dental restoration that can be fitted in a patient. In a preferred embodiment, the dental material is selected from the group consisting of dental material for preparing monolithic restorations, veneering material, material for pressing onto other materials, glazing material, framework material, bonding agent and stain.

The dental restoration is preferably selected from the group consisting of crown, bridge, framework, inlay, onlay, veneer, abutment, partial crown, facet, maxillary complete prosthesis, mandibular complete prosthesis, maxillary partial prosthesis and mandibular partial prosthesis.

In a preferred embodiment, the dental material comprises a glass, a glass ceramic, a feldspar ceramic, or a composition for preparing a glass, a glass ceramic, or a feldspar ceramic. It is particularly preferred that the glass, glass ceramic or composition for preparing the glass or glass ceramic is selected from the group consisting of glasses and glass ceramics based on lithium silicate, leucite, fluorapatite, oxyapatite, quartz, low quartz solid solution, high quartz solid solution, lithium alumosilicate and mixtures thereof, as well as compositions for preparing them.

In another preferred embodiment, the dental material is a glass ceramic, preferably selected from the group consisting of lithium silicate glass ceramic, leucite glass ceramic, fluorapatite glass ceramic, oxyapatite glass ceramic, quartz glass ceramic, high quartz solid solution glass ceramic, low quartz solid solution glass ceramic, and lithium alumosilicate glass ceramic. It is preferred that the total proportion of all crystal phases in the glass ceramic is at least 5 wt.-%.

It has surprisingly been found that the pigment can also be used for coloring dental materials that are glass ceramics. Even when there is a high proportion of crystal phases in the glass ceramic, the dental restoration can be colored with the pigment to the desired color tone.

It is also preferred that the dental material is a glass or a composition for preparing it, wherein the glass has a dilatometric softening point in the range of 400 to 1200 ° C., in particular 420 to 1150° C. and particularly preferably 450 to 1100° C. It is further preferred that the dental material is a glass or a composition for preparing it, wherein the glass has a dilatometric softening point in the range of 400 to 600° C., in particular 420 to 580° C. and particularly preferably 450 to 550° C., or in the range of 500 to 1200° C., in particular 600 to 1150° C. and particularly preferably 650 to 1100° C. For the purpose of the present invention, the dilatometric softening point of the glass can be determined according to DIN ISO 6872.

In a preferred embodiment, the dental material is a glazing material having a viscosity of more than 10^{2.5} P Pa·s at a temperature of 700° C., a viscosity of more than 10^{2.5} Pa·s at a temperature of 900° C., and/or a viscosity of less than 10⁹ Pa·s at a temperature of 1100° C.

In another preferred embodiment, the dental material has a color with an a*value in the range of 0 to 25, in particular 1 to 20, and particularly preferably 2 to 15.

It is also preferred that the dental material has a color with an a*value in the range from 0 to 13, preferably 1 to 11, particularly preferably 2 to 8. These a*values have been found to be particularly advantageous for imitating tooth color. It is also preferred that the dental material has a color with an a*value in the range of 5 to 25, preferably 5 to 20, particularly preferably 5 to 15. It has been found that these a*values are particularly suitable for imitating gingiva color. It is particularly preferred that, in addition to the preferred ranges of the a*values, the b* value is 5 to 35, in particular 5 to 25, and the L* value is 65 to 90, in particular 70 to 90.

In a preferred embodiment, the dental material contains the pigment in an amount of 0.005 to 20 wt.-%, in particular 0.01 to 16 wt.-%, and particularly preferably 0.05 to 10 wt.-%.

It is further preferred that the dental material contains the pigment in an amount of 0.005 to 5 wt.-%, in particular 0.01 to 2.5 wt.-% and particularly preferably 0.05 to 2 wt.-%. It has been found that these amounts are particularly suitable for imitating tooth color. It is also preferred that the dental material contains the pigment in an amount of 0.01 to 20 wt.-%, in particular 0.05 to 16 wt.-% and particularly preferably 0.1 to 10 wt.-%. These amounts have proven to be particularly suitable for imitating gingiva color.

The amount of pigment in the dental material can be determined by combining optical analyses of the microstructure, such as electron microscopy, with chemical analysis methods, such as energy dispersive X-ray spectroscopy. The proportion of pigment in the dental material is first determined volumetrically and then converted into a weight proportion, taking into account the densities of the dental material and the pigment. Crystal phases contained in the pigment can be determined by X-ray diffraction (XRD) analysis.

Moreover, the invention relates to the use of the dental material described above for the preparation of a dental restoration, and to the use of the pigment described above as a dental material, in particular as a dental colorant, or as a component of a dental material, in particular for coloring the dental material.

In a preferred embodiment, the dental restoration is selected from the group consisting of crown, bridge, framework, inlay, onlay, veneer, abutment, partial crown, facet, maxillary complete prosthesis, mandibular complete prosthesis, maxillary partial prosthesis and mandibular partial prosthesis.

The use according to the invention may comprise any process steps used for the preparation of a dental restoration. For example, the use may comprise giving the dental material the shape of the dental restoration.

In a preferred embodiment, the dental material, which is a glass ceramic, is pressed onto a framework. The framework preferably consists of metal, glass ceramic or oxide ceramic, in particular oxide ceramic based on ZrO₂ or Al₂O₃. In particular, the pressing involves the lost-wax method, and it is preferably used to apply to the framework a layer which is stained in a red color for imitating the gingiva.

In another preferred embodiment, the dental material is applied to a substrate, wherein the dental material is a glass having a dilatometric softening point in the range of 400 to 1200° C., preferably 420 to 1150° C., and particularly preferably 450 to 1100° C. The substrate preferably consists of metal, glass ceramic or oxide ceramic, in particular oxide ceramic based on ZrO₂ or Al₂O₃. It has been found that the pigment is stable also in these high-melting glasses at the temperatures required for processing. Therefore, these glasses can be applied to the substrate with the desired color effect.

In another preferred embodiment, the dental material is used as a stain. The stain can be applied to a substrate with a brush. Preferably, the stain is a glass with a dilatometric softening point in the range of 400 to 600° C., in particular 420 to 580° C. and particularly preferably 450 to 550° C., which is applied to a substrate, wherein the substrate is in particular based on an oxide ceramic and/or glass ceramic.

In another preferred embodiment, the dental material is used as a glazing material.

Preferably, the glazing material is a glass having a dilatometric softening point in the range of 500 to 1200° C., preferably 600 to 1150° C. and particularly preferably 650 to 1100° C. The glazing material is preferably applied to a substrate, wherein the substrate in particular consists of oxide ceramic or metal. It may be advantageous to first apply an opaquer material and optionally a dentin and incisal material before applying the glazing material to a substrate, in particular to a metal substrate. Preferably, the substrate is not densely sintered, in particular it is unsintered or presintered. It is preferred that the glazing material substantially does not penetrate into the substrate. It was found that the dental material of the invention can be used to apply coatings and red colors to not densely sintered substrates in a single step. With dental materials known in the prior art, coating and red coloring must be carried out in separate steps.

In a preferred embodiment of the use according to the invention, the dental material is a glazing material for glazing a non-densely sintered substrate, wherein the glazing material is applied to the non-densely sintered substrate and the substrate and the glazing material are subjected to a heat treatment in a temperature range extending from a first temperature T₁ to a second temperature T₂, which is higher than the first temperature T₁, wherein, at the temperature T₁, the glazing material has a viscosity of more than 10^{2.5} Pa·s, preferably more than 10^{4.0} Pa·s, in particular more than 10^{5.6} Pa·s and particularly preferably more than 10^{7.0} Pa·s and, at the temperature T₂, a viscosity of less than 10⁹ Pa·s, preferably less than 10⁷ Pa·s and in particular less than 10^{5.6} Pa·s.

Particularly preferably, the glazing material has, at a temperature of 950° C., a viscosity of more than 10^{2.5} Pa·s, in particular more than 10^{4.0} Pa·s, preferably more than 10^{5.6} Pa·s and particularly preferably more than 10^{7.0} Pa·s, at a temperature of 1300° C. a viscosity of more than 10^{2.5} Pa·s and preferably more than 10⁴ Pa·s and, at a temperature of 1450° C., a viscosity of less than 10⁹ Pa·s, preferably less than 10⁷ Pa·s and in particular less than 10^{5.6} Pa·s.

In another preferred embodiment, the glazing material has, at a temperature of 700° C., a viscosity of more than 10^{2.5} Pa·s, preferably more than 10^{4.0} Pa·s, in particular more than 10^{5.6} Pa·s and particularly preferably more than 10^{7.0} Pa·s, at a temperature of 900° C. a viscosity of more than 10^{2.5} Pa·s and preferably more than 10⁴ Pa·s and, at a temperature of 1100° C., a viscosity of less than 10⁹ Pa·s, preferably less than 10⁷ Pa·s and in particular less than 10^{5.6} Pa·s.

The viscosity of the glazing material can, in particular, be determined using a viscosity-temperature curve based on the Vogel-Fulcher-Tammann (VFT) equation:

$${log}_{10} ( η ) = A + \frac{B}{T - {T}_{0}}$$

n: dynamic viscosity at temperature T
A, B, T₀: substance-specific constants.

This equation is solved starting from at least three and preferably five pairs of values from characteristic temperatures determined experimentally by means of a dilatometer or heating microscope, respectively, and the associated viscosity values:

| Designation and measurement method | η (Pa · s) |
|---|---|
| T_{g} (glass transition point from dilatometer) | 12 |
| T_{d} (softening temperature from dilatometer) | 10 |
| T_{S} (softening point from heating microscope) | 5.6 |
| T_{HB} (hemisphere point from heating microscope) | 3.5 |
| T_{F} (flow point from heating microscope) | 2.1 |

The equation is solved by an approximation method according to the least-squares method.

In another preferred embodiment, the pigment is contained in a glazing material having the composition of the glazing material described in WO 2018/172544 A1 and corresponding U.S. Pat. No. 11,254,618 B2, which US patent is hereby incorporated by reference.

The invention also relates to a process for preparing a dental restoration, in which the dental material described above is processed, and in particular shaped, into the dental restoration, or the pigment described above is added to a restorative material and this restorative material is processed, and in particular shaped, into the dental restoration.

All the above-described embodiments of the dental material and the pigment, as well as the process steps are also correspondingly suitable or preferred for the process according to the invention for preparing a dental restoration.

The invention also relates to a process for preparing the pigment described above, in which a starting composition is calcined, wherein the starting composition comprises Al (OH)₃, Cr₂O₃, Z₂O₃ and optionally a flux agent, wherein Z is selected from the group consisting of Y, La and lanthanides, and during calcination the starting composition is heated from room temperature to the maximum temperature at an average heating rate of at least 5° C./min.

In a preferred embodiment, the starting composition contains one or more flux agents. Suitable flux agents are also referred to as mineralizers and they include fluorides and carbonates of calcium, sodium and barium. In a preferred embodiment, the composition contains CaF₂ or a combination of BaCO₃ and NaF as flux agent, in particular CaF₂ or a combination of BaCO₃ and NaF in a molar ratio of 5:1, and particularly preferably CaF₂. It has been found that particularly intense red colors can be obtained by using CaF₂, in particular in an amount of at least 3 wt.-%.

It is also preferred that the compositions contain a flux agent, in particular CaF₂, in an amount of 3.0 to 7.0 wt.-%, in particular 4.0 to 6.0 wt.-% and particularly preferably 4.5 to 5.5 wt.-%.

It is also preferred that the starting composition contains 0.1 to 6 mol-% Cr and in particular 1 to 4 mol-% Cr, based on the amount of Al.

In another preferred embodiment of the process, the starting composition contains one and preferably all of the following components in the given amounts:

| Component | Wt.-% |
|---|---|
| Z₂O₃ | 45 to 65, in particular 50 to 60, |
| Al(OH)₃ | 30 to 45, in particular 35 to 40, |
| Cr₂O₃ | 0.7 to 1.5, in particular 0.9 to 1.3, |
| CaF₂ | 3.0 to 6.0, in particular 4.5 to 5.5, |

wherein Z is selected from Y, La und lanthanides.

In a preferred embodiment, the components of the starting composition are comminuted and/or mixed prior to calcination, wherein the comminution may be performed manually or mechanically and is preferably carried out by grinding. The components of the starting composition may also be comminuted and mixed simultaneously or sequentially. It is also preferred that the comminuting and mixing results in a substantially uniform mixture of the components of the starting composition.

The comminution and mixing can be carried out dry or with the addition of a liquid. For example, the components of the starting composition can be dry mixed and ground together in a mortar (e.g., an agate mortar) and/or a mortar grinder (e.g., model RM200 from Retsch).

The components of the starting composition can also be mixed and simultaneously ground using ZrO₂ grinding beads with the addition of a liquid, preferably water and/or ethanol, in a mixing device that uses Dual Asymmetric Centrifuge (DAC) technology (e.g., SpeedMixer from Hauschild). The ZrO₂ grinding beads can then be removed from the obtained homogeneous suspension using a sieve, prior to subjecting the suspension to a drying step.

The process according to the invention comprises calcining the starting materials, which results in pigments with a red color. In a preferred embodiment, the calcination is carried out at a maximum temperature of 1200 to 1600° C., in particular 1250 to 1550° C. and particularly preferably 1300 to 1500° C.

Preferably, the average heating rate during calcination, in terms of heating from room temperature to the maximum temperature, is 0.1 to 150° C./min, in particular 0.5 to 100° C./min and particularly preferably 1 to 75° C./min.

In a preferred embodiment, calcination comprises first heating to a first temperature T₁ using a heating rate R₁ and then heating to the maximum temperature using a second heating rate R₂, wherein R₁ is greater than R₂, T₁ is 400 to 800° C. and preferably 500 to 700° C., R₁ is 1 to 100° C./min, preferably 2 to 75 ° C./min and particularly preferably 5 to 50° C./min, and R₂ is 1 to 100° C./min, preferably 2 to 75° C./min and particularly preferably 5 to 50° C./min.

It is furthermore preferred that the holding time of the calcination at the maximum temperature is 0.2 to 8 h, in particular 0.5 to 6 h.

Particularly preferably, the calcination is carried out at a temperature of 1000 to 1700° C., in particular 1000 to 1600 ° C., with a holding time of 0.2 to 8 h, in particular 0.5 to 6 h.

Surprisingly, it has been found that the heating rate and the holding time affect the color shade obtained by calcination. In particular, low heating rates, such as an average heating rate below 5° C./min, result in a color tone that is perceived as brown.

In a preferred embodiment, after calcination the pigments are ground and mixed again.

The invention is explained in more detail in the following with reference to examples.

### EXAMPLES

### Examples 1 to 11: Process for Preparing the Pigment

Pigments were prepared from the starting composition given in Table 1 by the process according to the invention.

TABLE 1

| Component | Wt.-% |
|---|---|
| Al(OH)₃ | 37.7 |
| Cr₂O₃ | 1.1 |
| Z₂O₃ | 56.2 |
| CaF₂ | 5.0 |

In this process, Z in component Z₂O₃ was selected for Examples 1 to 11 as given in Table 2.

TABLE 2

| Example | Z |
|---|---|
| 1 | Y |
| 2 | Er |
| 3 | Gd |
| 4 | Pr |
| 5 | Dy |
| 6 | La |
| 7 | Eu |
| 8 | Nd |
| 9 | Yb |
| 10 | Ho |
| 11 | Tm |

The starting compositions were mixed and comminuted using an agate mortar. Then they were calcined by heating them from room temperature to 600° C. within one hour (heating rate about 9.6° C./min) and then to 1300° C. within 2 hours (heating rate about 5.8° C./min). After a holding time of one hour at 1300 ° C., the compositions were allowed to cool freely, i.e., without controlling the temperature, to room temperature. It was observed that the composition, which previously had a green color, was given a red color during calcination.

The L*a*b* values of the calcined pigments were determined according to DIN 6174 using a spectrophotometer (CM 3700-D, Konica-Minolta). The values determined are given in Table 3.

TABLE 3

| Example | Z | L* | a* | b* |
|---|---|---|---|---|
| 1 | Y | 50.56 | 31.94 | 21.80 |
| 2 | Er | 52.08 | 26.02 | 15.52 |
| 3 | Gd | 46.16 | 20.11 | 13.06 |
| 4 | Pr | 41.16 | 8.72 | 5.98 |
| 5 | Dy | 48.57 | 23.09 | 12.73 |
| 6 | La | 63.61 | 7.13 | 5.85 |
| 7 | Eu | 45.87 | 11.05 | 7.83 |
| 8 | Nd | 44.34 | 12.48 | 7.69 |
| 9 | Yb | 52.56 | 25.42 | 15.83 |
| 10 | Ho | 51.09 | 23.75 | 15.72 |
| 11 | Tm | 56.89 | 24.84 | 15.63 |

### Example 12: Effect of the Heating Rate of Calcination on the Pigment Color

The effect of the heating rate used for calcination on the color of the calcined pigment was investigated. For this purpose, a starting composition according to Example 1 was prepared and calcined by heating it from room temperature to 1300 ° C. within 6 hours (heating rate about 3.6° C./min). After a holding time of 6 hours at 1300° C., the composition was allowed to cool to 900° C. within 2 hours and then freely to room temperature. Thus, the temperature profile used for this example was in accordance with a commonly used process for calcining pigments. During calcination, the composition was given a brown color. Determination of the color of the pigment according to DIN 6174 using a spectrophotometer (CM 3700-D, Konica-Minolta) resulted in an L* value of 40.98, an a*value of 15.65 and a b* value of 9.79.

### Examples 13 to 23: Use of the Pigment in Low-Sintering Glazes

The pigments were used to color a glass which has a dilatometric softening point of 491 ° C. and is intended as a glazing material. The composition of the glass is given in Table 4.

TABLE 4

| Component | Wt.-% |
|---|---|
| SiO₂ | 65.01 |
| K₂O | 4.55 |
| Na₂O | 7.45 |
| Li₂O | 3.30 |
| CaO | 1.80 |
| SrO | 2.42 |
| Al₂O₃ | 5.02 |
| MgO | 0.45 |
| ZrO₂ | 0.61 |
| ZnO | 3.50 |
| B₂O₃ | 4.02 |
| F | 1.30 |

For preparing Examples 13 to 23, the powdered glass was colored with 1 wt.-% pigment in each case. The lanthanum- or lanthanide-containing pigments of Examples 1-6 and 8-11 were used for the preparation of Examples 13 to 22. The powdered glass of Example 23 was colored with 1 wt.-% of the brown pigment of Example 12. The pigments used for preparing the corresponding Examples are given in Table 5.

The colored glass powders of Examples 13 to 23 were fired at a temperature of 710° C. in accordance with the dental use. Then, the L*a*b* values of the colored glasses were determined using a spectrophotometer (CM 3700-D, Konica Minolta) according to DIN 6174. The L*a*b* values measured are given in Table 5.

TABLE 5

| | | Pigment of | | | |
|---|---|---|---|---|---|
| Example | Z | Example | L* | a* | b* |
| 13 | Y | 1 | 45.35 | 35.92 | 23.40 |
| 14 | Er | 2 | 45.91 | 34.20 | 20.49 |
| 15 | Gd | 3 | 42.55 | 29.11 | 17.30 |
| 16 | Pr | 4 | 39.80 | 13.23 | 8.96 |
| 17 | Dy | 5 | 42.42 | 28.12 | 15.00 |
| 18 | La | 6 | 53.71 | 16.58 | 9.82 |
| 19 | Nd | 8 | 41.47 | 19.46 | 10.85 |
| 20 | Yb | 9 | 45.99 | 34.69 | 21.30 |
| 21 | Ho | 10 | 44.27 | 30.98 | 19.34 |
| 22 | Tm | 11 | 49.79 | 34.89 | 21.42 |
| 23 | Y | 12 | 42.87 | 36.61 | 22.57 |

An intense red color was obtained in the glass by using the pigments. There was no clear difference perceptible to the naked eye between the colors of glasses 13 and 23. This is surprising because the color of these Y-containing pigments in powder form was clearly different.

### Examples 24 to 32: Use of the Pigment in a Fluorapatite Glass-Ceramic

The pigments were also used to color a fluorapatite glass-ceramic having the composition given in Table 6. For this purpose, an appropriate e mixture of raw materials, such as oxides, carbonates, phosphates and halides, was melted at a temperature of 1500° C. for 1 to 3 h and then poured into water to produce a frit. The frit was dried, ground, and formed into a tempered cake. The tempered cake was held for 1 h at a temperature of 1020° C., then quenched in water, dried, and ground into a powdered glass ceramic.

TABLE 6

| Component | Wt.-% |
|---|---|
| SiO₂ | 57.68 |
| K₂O | 9.81 |
| Na₂O | 6.37 |
| CaO | 2.72 |
| Al₂O₃ | 16.46 |
| B₂O₃ | 0.25 |
| ZrO₂ | 2.17 |
| CeO₂ | 0.51 |
| F | 0.57 |
| ZnO | 1.20 |
| TiO₂ | 1.05 |
| P₂O₅ | 1.21 |

5 For each of Examples 24 to 33, the powdered glass ceramic was colored with 1 wt.-using the lanthanum- or lanthanide-containing pigments of Examples 2-6 and 8-11. The pigments used for the preparation of each Example are given in Table 7.

TABLE 7

| | | Pigment | | | |
|---|---|---|---|---|---|
| | | of | | | |
| Example | Z | Example | L* | a* | b* |
| 24 | Er | 2 | 52.97 | 30.85 | 18.95 |
| 25 | Gd | 3 | 46.87 | 29.36 | 17.69 |
| 26 | Pr | 4 | 41.87 | 13.57 | 8.39 |
| 27 | Dy | 5 | 49.44 | 27.35 | 14.98 |
| 28 | La | 6 | 57.88 | 14.62 | 8.90 |
| 29 | Nd | 8 | 43.84 | 19.28 | 9.96 |
| 30 | Yb | 9 | 52.99 | 31.40 | 20.56 |
| 31 | Ho | 10 | 50.86 | 28.50 | 19.00 |
| 32 | Tm | 11 | 55.70 | 30.20 | 18.94 |

Intense red colors were obtained in the glass ceramic by using the pigments.

### Examples 33 to 36: Heat Stability in Fluorapatite Glass Ceramics

The powdered fluorapatite glass ceramic used in Examples 24 to 33 was employed to prepare Examples 33 to 36. For preparing Examples 33 and 34 the powdered glass ceramic was in each case colored with 1 wt.-% of the pigment of Example 1. For preparing Examples 35 and 36, in each case 1 wt.-% of the pigment used for preparing Example 17, the color of which is perceived as brown, was added to the powdered glass ceramic.

In accordance with the dental use, the colored glass ceramic powders were fired in a dental furnace for 1 min, wherein a temperature of 1080° C. for each of Examples 33 and 35 and a temperature of 1170° C. for each of Examples 34 and 36 was used.

The L*a*b* values of the glass ceramics were determined using a spectrophotometer (CM 3700-D, Konica Minolta) according to DIN 6174. The firing temperatures used as well as the color values measured are given in Table 8.

TABLE 8

| | Firing | | | |
|---|---|---|---|---|
| Example | temperature (° C.) | I* | a * | b* |
| 33 | 1080 | 50.56 | 33.19 | 21.78 |
| 34 | 1170 | 50.82 | 32.89 | 20.99 |
| 35 | 1080 | 48.26 | 34.57 | 21.69 |
| 36 | 1170 | 48.21 | 34.24 | 20.88 |

Comparing the a*values of Examples 33 and 34 or 35 and 36, respectively, reveals that a higher firing temperature does not result in a reduction of the a*value. This demonstrates that the pigments exhibit very good stability even at a firing temperature of 1170° C. in the high-firing glass ceramic.

As already described for the use in the glasses of Examples 13 and 23, it is possible to bring about intense red colors in glass ceramics with pigments which exhibit a red color in powder form as well as with pigments which exhibit a brown color in powder form. When comparing glass ceramics 33 and 35 or 34 and 36, respectively, no clear color difference was perceptible to the naked eye.

1. A dental material comprising
a pigment,
wherein the pigment comprises Al, Cr and Z, and Z is selected from the group consisting of Y, La, lanthanides and mixtures thereof.

2. The dental material according to claim 1,
in which the pigment comprises Al and Z in a molar ratio of 0.7:1 to 1:0.7.

3. The dental material according to claim 1,
in which the pigment comprises Z, Al and Cr in a molar ratio corresponding to the formula ZₓAl_{2-x-y}Cr_{y}O₃,
wherein x is 0.8 to 1.2, and
y is 0.001 to 0.5.

4. The dental material according to claim 1,
in which the main crystal phase of the pigment has a perovskite crystal structure.

5. The dental material according to claim 1,
in which Z is selected from the group consisting of Y, Er, Pr, Gd, Dy, Eu, Nd, Yb, Ho and Tm.

6. The dental material according to claim 1,
in which the pigment has an average particle size d₅₀ of 0.05 to 50 μm.

7. The dental material according to claim 1,
which is present in the form of a powder, a blank or a dental restoration.

8. The dental material according to claim 1,
which comprises a glass, a glass ceramic, a feldspar ceramic or a composition for preparing a glass, a glass ceramic or a feldspar ceramic.

9. The dental material according to claim 8,
in which the glass, the glass ceramic or the composition for preparing the glass or glass ceramic is selected from the group consisting of glasses and glass ceramics based on lithium silicate, leucite, fluorapatite, oxyapatite, quartz, low quartz solid solution, high quartz solid solution, lithium alumosilicate and mixtures thereof as well as compositions for preparing them.

10. The dental material according to claim 1,
which has a color with an a*value in the range of 0 to 25, or in the range of 5 to 25.

11. The dental material according to claim 1,
which comprises the pigment in an amount of 0.005 to 20 wt.-%.

12. A process for preparing a dental restoration,
in which the dental material according to claim 1 is processed and shaped into a dental restoration or the pigment is added to a restorative material and the restorative material is processed and shaped into a dental restoration.

13. A process for preparing the pigment defined in claim 1,
in which a starting composition is calcined,
wherein the starting composition comprises Al (OH)₃, Cr₂O₃, Z₂O₃ and optionally a flux agent, wherein Z is selected from Y, La und lanthanides, and
during calcination the starting composition is heated from room temperature to the maximum temperature using an average heating rate of at least 5° C./min.

14. The process according to claim 15,
in which the starting composition comprises at least one of the following components in the indicated amounts:

| Component | Wt.-g |
|---|---|
| Z₂O₃ | 45 to 65, |
| Al(OH)₃ | 30 to 45, |
| Cr₂O₃ | 0.7 to 1.5, |
| CaF₂ | 3.0 to 6.0, |

wherein Z is selected from Y, La und lanthanides.

15. The process according to claim 13,
in which the calcination comprises heating to a first temperature T₁ using a heating rate R₁ and then heating to the maximum temperature using a second heating rate R₂, wherein R₁ is greater than R₂, T₁ is 400 to 800° C., R₁ is 1 to 100 ° C./min, and R₂ is 1 to 100° C./min.
